(19) United States
(12) Patent Application Publication
(10) Pub. No.: US 2009/0137965 A1
(43) Pub. Date: May 28, 2009

(54) Apparatus for Operating Syringe Piston

(76) Inventors: Seok-Jung Kim, Gyeonggi-Do (KR); Cheong-Ho Chang, Seoul (KR); Jae-Deog Jang, Seoul (KR); Hyun-Shin Park, Seoul (KR); Sae-Bom Lee, Seoul (KR); Kyoung-Phil Byun, Gyeonggi-Do (JP)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/226,316

(22) PCT Filed: Jun. 2, 2006
(86) PCT No.: PCT/KR2006/002117

§ 371 Date: Oct. 15, 2008

(30) Foreign Application Priority Data
Apr. 21, 2006 (KR) 10-2006-0036206

(51) Int. Cl. A61M 5/315

(52) U.S. Cl. 604/208

(57) ABSTRACT

An apparatus for operating a syringe piston is developed, which is comprised of: a syringe mounting section for fixedly holding a syringe, a thread hole formed at an upper head part of the syringe mounting section, and a piston press unit having a threaded rod, which is inserted into the thread hole so that the syringe piston press unit can move up and down along the thread hole, a locking slot for fixedly inserting and retaining the medicine filled syringe, a semi-cylindrical tube to assemble with the syringe mounting section. The syringe filled with medical cement is attached to the syringe mounting section and the syringe piston is moved downward with no reverse movement when the piston press unit is rotated, so the medical cement contained in the syringe is injected with a constant pressure, so that a predetermined amount of medical cement is precisely injected into a patient's body.

### BACKGROUND OF THE INVENTION

1. Field of the Invention

The present invention relates to an apparatus for operating a syringe piston. More particularly, it is comprised of a syringe mounting section for holding a medicine filled syringe and a piston press unit for operating a syringe piston without reverse action while the piston is depressing, so that the medicine contained in the syringe can be injected into a patient's body with constant pressure, ensuring that an accurate amount of medicine is delivered.

2. Related Prior Art

As generally known in the art, a syringe is a device for injecting a liquid medicine into a tissue of a living body. The syringe mainly includes a needle inserted into the tissue of the living body, a glass cylinder containing the liquid medicine, and a piston for applying pressure to the liquid medicine. Recently, disposable plastic syringes have been extensively used in order to avoid diseases derived from serum infection.

Meanwhile, a medical treatment employing medical cement has been proposed to cure lesions, such as the fracture, amputation or necrosis of a bone. According to the above medical treatment employing the medical cement, an incision is made so that the surgeon can see the injured bone with the naked eye, and then the medical cement is injected into the lesioned part. Otherwise, the surgeon checks the lesioned part by observing the bone using an imaging device, and then injects the medical cement into the tissue of the patient using the syringe.

However, the conventional syringe is rarely used for this type of procedure due to the following reason: when the medical cement is injected into the tissue of the patient, a relatively great pressure must be applied to the piston due to the physical properties of the medical cement. However, it is very difficult to manually apply a constant pressure to the piston, thus it is also very difficult to inject a precise amount of medical cement into the tissue of the patient.

### SUMMARY OF THE INVENTION

Accordingly, the present invention has been made to solve the above-mentioned problems occurring in the prior art, and a first object of the present invention is to provide an apparatus for operating a syringe piston, which includes a syringe mounting section for securely mounting a syringe and a piston press unit for pressing the syringe piston with a constant pressure.

A second object of the present invention is to provide an apparatus for operating a syringe piston, in which a syringe filled with medical cement is attached to a syringe mounting section and the syringe piston is moved downward without any reverse motion when a user rotates a piston press unit having a threaded rod, so medical cement contained in the syringe can be extruded from the syringe with a constant pressure, so that a predetermined amount of medical cement can be precisely injected into a human body.

A third object of the present invention is to provide an apparatus for operating a syringe piston, which can significantly improve the medical technique.

A fourth object of the present invention is to provide an apparatus for operating a syringe piston, which can significantly improve the quality and reliability of procedures, thereby providing consumers with high satisfaction and a good impression.

In order to accomplish the above objects, according to the present invention, there is provided an apparatus for operating a syringe piston, the apparatus comprising: a syringe mounting section for fixedly mounting a syringe, in which a screw hole is formed at an upper head part of the syringe mounting section; and a piston press unit having a threaded rod, which is threaded into the screw hole so that the syringe piston press unit can move up and down along the screw hole, wherein the piston press unit presses a syringe piston with a constant pressure such that a predetermined amount of contents is precisely injected into a human body from the syringe.

### BRIEF DESCRIPTION OF DRAWINGS

FIG. 1 is a perspective view illustrating an apparatus for operating a syringe piston according to the present invention.

FIG. 2 is a front view illustrating an apparatus for operating a syringe piston according to the present invention.

FIG. 3 is a side view illustrating an apparatus for operating a syringe piston according to the present invention.

FIG. 4 is a view illustrating an apparatus for operating a syringe piston according to the present invention, in which a piston press unit has not yet moved downward.

FIG. 5 is a view illustrating an apparatus for operating a syringe piston according to the present invention, in which a piston press unit moves downward while pushing a syringe piston in the downward direction.

FIG. 6 is a perspective view illustrating an operational state of an apparatus for operating a syringe piston according to the present invention.

### DETAILED DESCRIPTION OF THE PREFERRED EMBODIMENTS

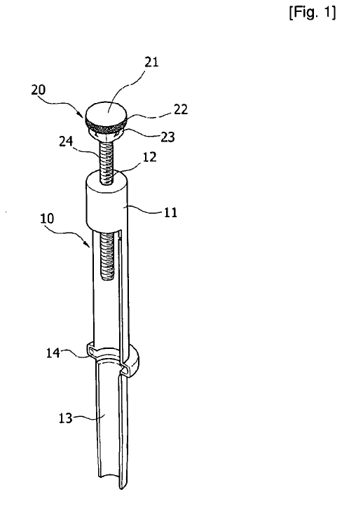

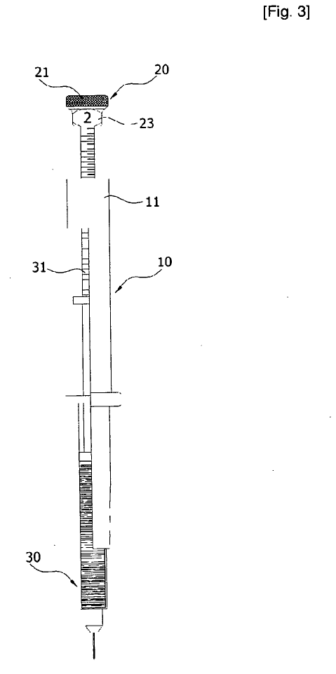

Hereinafter, an apparatus for operating a syringe piston according to a preferred embodiment of the present invention will be described with reference to FIGS. 1 to 6.

In the following description of the present invention, a detailed description of known functions and configurations incorporated herein will be omitted when it may make the subject matter of the present invention rather unclear.

In addition, terms used in the following description of the present invention are prepared in view of functions thereof, so they will be changed depending on the intention of manufacturers or custom. Thus, definition of the terms must be determined based on the whole content of the specification.

The present invention provides an apparatus for operating a syringe, which is comprised of a syringe mounting section 10 for securely holding a syringe 30 and a piston press unit 20 for operating a syringe piston 31 with a constant pressure. The syringe 30 filled with medical cement is placed into the syringe mounting section 10 for operating the syringe piston 31 downward without any reverse motion. When a screw of the piston press unit 20 is rotated, the medicine contained in the syringe 30 can be injected into the patient from the syringe 30 with a constant pressure. So, a precise amount of medicine can be injected into a patient's body. Hereinafter, the structure of the present invention will be described in more detail.

The syringe mounting section 10 is integrally formed with a lower portion of the semi-cylindrical tube 13 for detachably mounting the syringe 30 while allowing an operator to see the inside of the syringe mounting section 10 from the exterior. The diameter “A” of an intermediate portion of the semi-cylindrical tube 13 is larger than the diameter “a” of a lower tip portion of the semi-cylindrical tube 13, so the syringe 30 is press-fitted into the semi-cylindrical tube 13 while generating a “click” sound, so that the syringe 30 is prevented from easily falling off of the semi-cylindrical tube 13.

In addition, the syringe mounting section 10 is formed with a locking slot 14, into which an upper end portion of the syringe is fixedly inserted.

A handle 21 is provided at an upper end portion of the piston press unit 20. The handle 21 is provided at an outer peripheral surface thereof with a knurled section 22 so as to allow a user to easily rotate the handle 21.

In addition, a polygonal mark section 23 is provided at a lower end portion of the handle 21. Figures, characters or symbols are formed in the polygonal mark section 23 so as to allow the user to rotate the piston press unit 20 corresponding to an amount of medical cement to be discharged from the syringe 30.

In particular, according to the present invention, the pitch of a threaded rod 24 and a screw hole 12 is established such that 100 ml of contents can be discharged from the syringe 30 as the user rotates the piston press unit 20 one time.

Meanwhile, the structure of the present invention can be variously modified and changed.

In addition, the present invention is not limited to the above embodiment, and those skilled in the art will appreciate that various modifications, additions and substitutions are possible, without departing from the scope and spirit of the invention as disclosed in the accompanying claims.

Hereinafter, the operation and effect of the present invention having the above structure will be described.

First, the user fills the syringe 30 with contents. At this time, the contents refer to a liquid medicine, which is injected into a tissue of a living body. According to the present invention, the contents preferably include medical cement used for curing lesions, such as the fracture, amputation or necrosis of a bone.

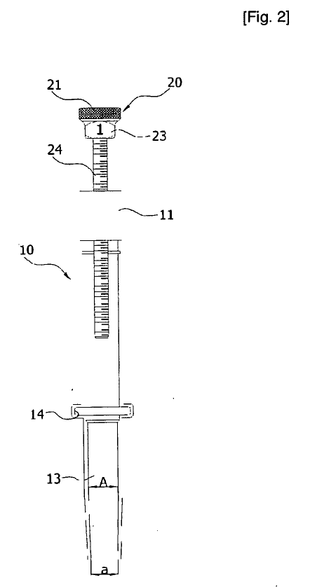

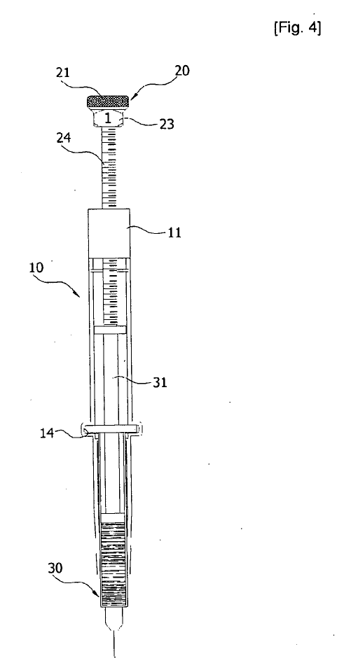

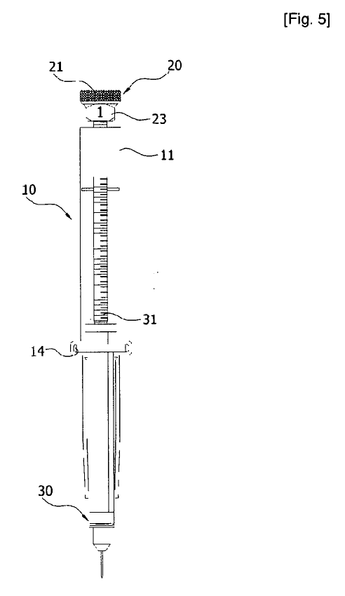

As shown in FIGS. 4 and 5, the syringe 30 filled with the medical cement is attached to the syringe mounting section 10. Since the syringe mounting section 10 is formed as a semi-cylindrical tube 13, about half of the syringe 30 is accommodated in the syringe mounting section 10. In addition, as shown in FIG. 2, since the diameter “A” of the intermediate portion of the semi-cylindrical tube 13 is larger than the diameter “a” of the lower tip portion of the semi-cylindrical tube 13, the syringe 30 is press-fitted into the semi-cylindrical tube 13 while generating a “click” sound, so that the syringe 30 can be prevented from being easily separated from the semi-cylindrical tube 13.

When the syringe 30 is press-fitted into the semi-cylindrical tube 13, an upper protrusion of the syringe 30 is firmly fitted into the locking slot 14. Thus, the syringe 30 can be prevented from sliding out of the semi-cylindrical tube 13 when the syringe piston 31 is pressed with a constant pressure.

After the syringe 30 filled with the medical cement has been press-fitted into the syringe mounting section 10, the piston press unit 20 is coupled to the syringe mounting section 10.

That is, the threaded rod 24 is threaded into the screw hole 12 formed at the center portion of an upper head part 11 of the syringe mounting section 10. At this time, as mentioned above, the pitch of the threaded rod 24 is predetermined such that 100 ml of contents can be discharged from the syringe 30 as the user rotates the piston press unit 20 one time.

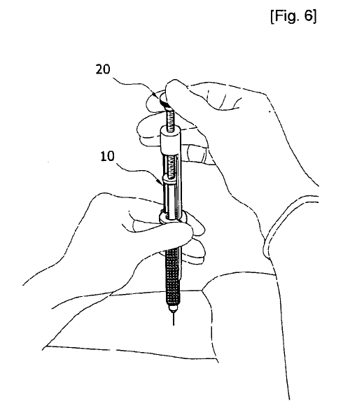

In detail, if the user rotates the piston press unit 20, a lower tip portion of the piston press unit 20 makes contact with an upper end portion of the syringe piston 31 as shown in FIGS. 4 and 6. In this state, if the user further rotates the piston press unit 20, the piston press unit 20 moves down together with the syringe piston 31 so that the contents (medical cement) of the syringe 30 are extruded from the syringe 30. Thus, the medical cement can be injected into a tissue of a patient.

According to the present invention, the syringe 30 filled with medical cement is attached to the syringe mounting section 10 and the syringe piston 31 is moved downward with no reverse motion when the user rotates the piston press unit 20 having the threaded rod, so medical cement contained in the syringe 30 can be extruded from the syringe 30 with a constant pressure, so that a predetermined amount of medical cement can be precisely injected into a human body. Thus, the present invention can significantly improve the medical technique.

Meanwhile, since the handle 21 is provided at the outer peripheral surface thereof with the knurled section 22, the user can easily rotate the handle 21 without slipping from the handle 21.

In addition, since the polygonal mark section 23 is provided at the lower end portion of the handle 21, the user can properly rotate the piston press unit 20 corresponding to the amount of medical cement to be discharged. That is, the user can adjust the rotation degree of the piston press unit 20 based on figures marked on the polygonal mark section 23. For instance, if it is necessary to inject 50 ml of contents into the tissue of the patient, the user simply rotates the piston press unit 20 by an angle of 180 degrees (that is, the user rotates the piston press unit 20 until a figure “3” is located in the front of the polygonal mark section 23).

The apparatus for operating the syringe piston according to the present invention can be mainly used for medical purposes. Of course, the apparatus of the present invention can also be used at home and in industrial fields.

As described above, the present invention provides the apparatus for operating the syringe piston, which includes the syringe mounting section for securely mounting the syringe and the piston press unit for pressing the syringe piston with a constant pressure. In particular, the syringe filled with medical cement is attached to the syringe mounting section and the syringe piston is moved downward with no reverse movement when the user rotates the piston press unit having the threaded rod, so medical cement contained in the syringe can be extruded from the syringe with a constant pressure, so that a predetermined amount of medical cement can be precisely injected into a human body. As a result, the apparatus for operating the syringe piston according to the present invention can significantly improve quality and reliability of procedures, thereby providing consumers with high satisfaction and a good impression.

What is claimed is:

1-6. (canceled)

7. An apparatus for operating a syringe piston, the apparatus comprising:
a syringe mounting section (10) integrally forming an upper head part (11) with a threaded hole (12) for placing a medicine filled syringe thereof,
a semi-cylindrical tube (13) to be assembled with the syringe mounting section (10), a tip portion diameter (a) of said semi-cylindrical tube (13) has smaller diameter than the diameter (A) of the upper portion thereof,
a locking slot (14) disposed between the syringe mounting section (10) and the semi-cylindrical tube (13) for fixedly inserting and retaining the medicine filled syringe, and
a piston press unit (20) having a threaded rod (24), which is screw-coupled to the threaded hole (12), so that the piston press unit (20) is movable along the threaded hole (12), while the syringe piston is depressed with a constant pressure for injecting precise amount of medicine to a patient body.

8. The apparatus as claimed in claim 7, wherein said syringe mounting section (10) is formed with a semi-cylindrical cutout for detachably placing the syringe, to be visual the placed syringe thereof.

9. The apparatus as claimed in claim 8, wherein said semi-cylindrical tube has a diameter (A) at an upper portion is larger than a diameter (a) of the tip portion thereof, so the syringe is snapped-fit into the semi-cylindrical tube to prevent falling off.

10. The apparatus as claimed in claim 7, wherein said piston press unit (20) further comprises a handle (21), an anti-slip knob section (22) formed at an outer peripheral surface of handle (21) for easily turning the handle.

11. The apparatus as claimed in claim 10, wherein said handle (21) further comprises a polygonal section (23) formed at a lower end portion thereof, and figures are marked on the polygonal section (23) to accurately calibrate injecting amount of the medicine.
